(19) United States
(12) Patent Application Publication
(10) Pub. No.: US 2012/0296293 A1
(43) Pub. Date: Nov. 22, 2012

(54) Bariatric absorbent diaper

(75) Inventors: Alan A. Clifford, Marion, OH (US); Andrew J. Szypka, Curtice, OH (US)

(73) Assignee: Principle Business Enterprises, Inc.

(21) Appl. No.: 13/373,327

(22) Filed: Nov. 10, 2011

Related U.S. Application Data
(60) Provisional application No. 61/456,790, filed on Nov. 12, 2010.
(60) Provisional application No. 61/456,791, filed on Nov. 12, 2010.

(51) Int. Cl. A61F 13/493; A61L 15/42; A61F 13/49

(52) U.S. Cl. 604/367; 604/385.01; 604/385.16

(57) ABSTRACT

A disposable, absorbent type-style diaper for bariatric incontinent users includes a non-woven top sheet, an absorbent core containing fluff and super absorbent polymer, a fluid-impervious backsheet (microporous) that covers at minimal the absorbent core at it's widest point, a non-woven backsheet layer that is glued to the fluid-impervious backsheet and the overlaying non-woven top sheet. The diaper will also contain standing leg-cuffs and elastomeric side panels (air-permeable). The design of the diaper is such that the rise of the diaper is significantly longer than normal diapers. This feature allows the diaper to fit bariatric wearers higher in the front belly area and maintains acceptable coverage on the backside. The non-woven top sheet and the non-woven backsheet are coated with an antimicrobial/anti-fungal compound that inhibits bacterial/fungi to grow on the surfaces of the diaper.

### CROSS REFERENCE TO RELATED APPLICATIONS

This application claims the benefit of U.S. provisional patent application No. 61/456,790 filed on Nov. 12, 2010 and U.S. provisional patent application No. 61/456,791 filed on Nov. 12, 2010.

### FIELD OF THE INVENTION

The present invention relates to an absorbent bariatric-style diaper of the kind which is intended for one-time use only and comprises of a non-woven top sheet, an absorbent core containing fluff and super absorbent polymer, a fluid-impervious backsheet (micro-porous) that covers a minimal the absorbent core at it's widest point, a non-woven backsheet layer that is glued to the fluid-impervious backsheet and the overlaying non-woven top sheet.

The diaper will also contain standing leg-cuffs and elastomeric side panels (air-permeable) that are attached to the non-woven wings by a conventional fixation method (ultrasonic boding, hotmelt adhesive, or heat seal bond). The problem with current Bariatric Briefs is that the design addresses waist size (girth) but does not address the issue of product length (rise). Product length (rise) is as important as waist size in that, as a person's body size increases in the waist dimension, the distance between the center line of the crotch and the normal beltline also increases, typically proportionately to waist increase. This increase in the crotch to beltline measurement can be symmetrical or asymmetrical, dependant on body build. As a result, caregivers using the current design product are often forced to rotate the product forward or backward to accommodate body type in an attempt to provide adequate coverage of the pubic area or posterior. This rotation repositions the core relative to the body and can compromise the products ability to provide proper incontinence protection. The improved design provides additional product length (rise) which allows the product to be positioned on the body of an obese person in the same manner as a non-obese person—this eliminates the rotation of the product on the body and allows for optimal positioning of the absorbent core to relative to the body. This increase in rise is accomplished by adding length to the front and back of the product. In doing so, the product now fits on a person in a near normal fashion relative to the person's beltline. In instances of severe obesity this results in the front of the product extending up and under the wearers pannis. This places plastic between skin folds which can cause an increase in skin temperature and increased skin moisture. These conditions can result in increased skin breakdown. To address this issue, this product has been designed with all breathable components in this region to optimize air flow and it has been coated with an antimicrobial/antifungal to help mange skin health in the skin fold areas.

The diaper design also incorporates 406 mm long elastic panels containing mechanical hook-style tape tabs which forms a large waist opening . . . 100+(2540 mm+) inch waist size. Other features of the diaper include microporous film (fluid-impervious back sheet), perforated/aperatured elastic side panels, and air-permeable non-woven panels which form 100% breathable diaper. The last feature is the non-woven top sheet and the non-woven backsheet are coated with an antimicrobial/anti-fungal compound that inhibits bacterial/fungi (micro-organisms) to grow on the surfaces of the diaper. This inhibits skin breakdown and odor inside the diaper and also on the outside of the diaper where the diaper comes in contact with the wearer's skin folds, pannis, and hip folds. Typically, these are the areas where odor is produced and skin breakdown occurs due to the moist environment. Also note that stretchable composites are currently used in diapers, mainly as extensions for tape tabs for improved fit. These composites are typically produced using an elastomeric film that is sandwiched between two layers of non-woven. Most commercial grades are non-porous and not air-permeable.

### BACKGROUND OF THE INVENTION

Currently, there are many Adult Incontinence absorbent-disposable diapers being produced and sold in the U.S. These designs are specifically made for the general population and sizes are broken out into Small, Medium, Large, and X-Large. These diapers are not designed for the bariatric-sized incontinent person. Also, these wearers have unique needs regarding fit, odor control, absorbency, and moisture control/antimicrobial properties. Special needs for fit relates to the coverage of the front and back of obese/bariatric wearers. These wearers have skin folds, pannis and hip folds not associated with standard incontinent wearers. These folds and the pannis increase the size of the coverage needed by the diaper in the front and standard diapers will not address this issue. A higher rise/larger waist diaper-design is required. Current practice is to rotate standard diapers forward to cover this larger skin area but then leaves the backside unprotected. Pressure ulcers are common due to incorrect fitting diapers. Skin care is also very important for obese/bariatric wearers. Wearers of upwards of 750 lbs required special skin-management designed garments to maintain breathability, reduce moisture, and provide a clean environment to inhibit micro-organisms. Moisture related skin issues such as: moisture associated dermatitis, incontinent associated dermatitis, candidiasis, cellulites, infection, odor, erythrasma, MRSA, and VRE. To help provide a safe and clean environment for the obese/bariatric wearer, a 100% breathable diaper-design with antimicrobial properties (inside and outside) was designed.

### SUMMARY OF THE INVENTION

The present invention, however, provides an absorbent disposable bariatric diaper of the kind defined in the introduction which will effectively enable the diaper to fit obese/bariatric wearers effectively, especially over the front area of the body and will not require rotating the diaper forward to provide frontal coverage. It will also provide highly absorbent and dry containment of body waste and fluids without leakage. This diaper design will also provide a 100% breathable environment that inhibits micro-organisms on its surface thus providing a clean environment in ski folds/etc. . . . According to one embodiment, an absorbent bariatric-style diaper of the kind which is intended for one-time use only and comprises of a spunbond polypropylene non-woven top sheet, an absorbent core containing fluff and super absorbent polymer, a fluid-impervious micro-porous polypropylene film backsheet that covers at minimal the absorbent core at it's widest point, a spunbond polypropylene non-woven backsheet layer that is glued with hotmelt to the fluid-impervious backsheet and the overlaying non-woven top sheet. The diaper will also contain standing leg-cuffs and elastomeric side panels (air-permeable) that are attached to the non-woven wings by a conventional fixation method (ultrasonic bonding, hotmelt adhesive, or heat seal bond). The design of the diaper is such that the rise of the diaper (diaper section from bottom of front wing to front top of diaper) is significantly longer than normal diapers (approx. 200-250 mm longer). This feature allows the diaper to fit bariatric wearers higher in the front belly area and maintains acceptable coverage on the backside. Standard normal diapers maintain coverage in the rear but fall short in coverage on the belly/front side, thus causing caregivers to rotate the diaper to the front to limit leakage from the front. The diaper design also incorporates from about 5 inches to about 20 inches long elastic panels containing mechanical hook-style tape tabs which forms a large waist opening of 100 inches+(2540 mm+) inch waist size. Other features of the diaper includes microporous film (fluid-impervious back sheet), perforated/aperatured elastic side panels. And air-permeable non-woven panels which form a 100% breathable diaper. A very important feature to be expanded on is the elastic-breathable side panels. The elastic side panels are made with an elastomeric film that is sandwiched between two non-woven layers. These layers are typically bonded together by thermal bonds (embossed-type patterns) which allows the composite to be stretchable and retractable. Our panels are produced in a way that are fully stretchable but are also perforated/aperatured to allow air-flow. Air permeability is high, where air permeability tested per ASTM D-737 is greater than 10 m³/m²/min. A typical design for the invention has an elastic composite for the elastic side panels containing 30 holes/inch² where holes are approx. 750-850 microns in size in a semi-stretched mode (approx. 10-25% condition). The last feature is the non-woven top sheet and the non-woven backsheet are coated with an antimicrobial/anti-fungal compound (AEGIS) that inhibits bacterial/fungi (micro-organisms) to grow on the surfaces of the diaper. This inhibits skin breakdown and odor inside the diaper and also on the outside of the diaper where the diaper comes in contact with the wearer's skin folds, pannis, and hip folds. Typically, these are the areas where odor is produced and skin breakdown occurs due to the moist environment.

Other objects and advantages of the present invention will become apparent to those skilled in the art upon a review of the following detailed description of the preferred embodiments and the accompanying drawings.

### BRIEF DESCRIPTION OF THE DRAWINGS

FIG. 1 is a plan view of the front of the diaper of the present invention.

FIG. 2 is a plan view of the back of the diaper.

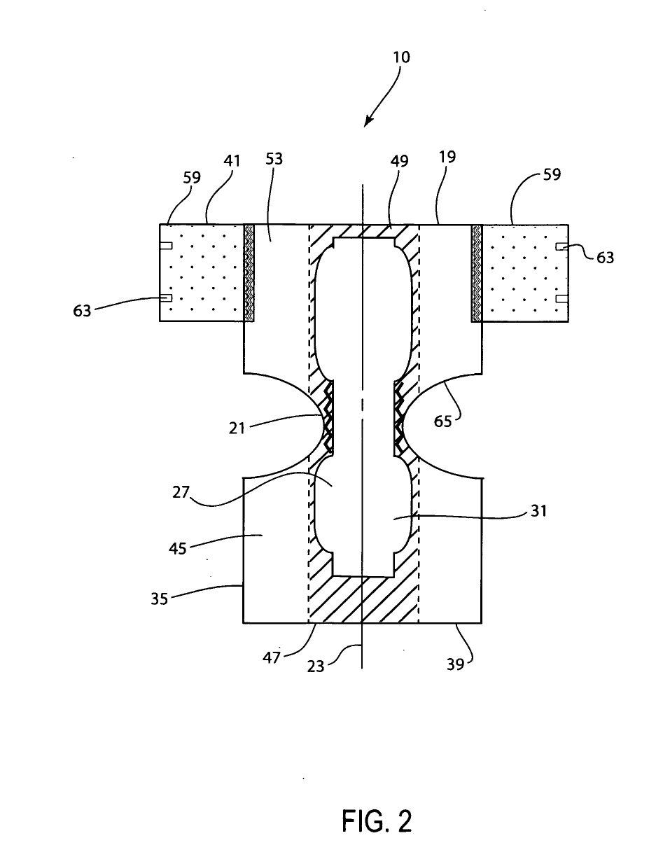

FIG. 3 is a cross sectional view of the diaper of the present invention.

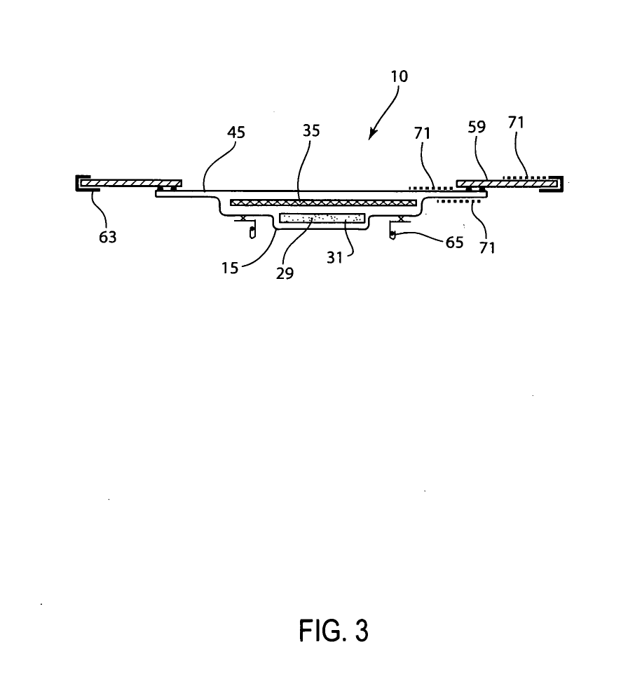

FIG. 4 is an exploded side elevation view of the elastic side panels of the invention.

### DETAILED DESCRIPTION OF THE PREFERRED EMBODIMENT(S)

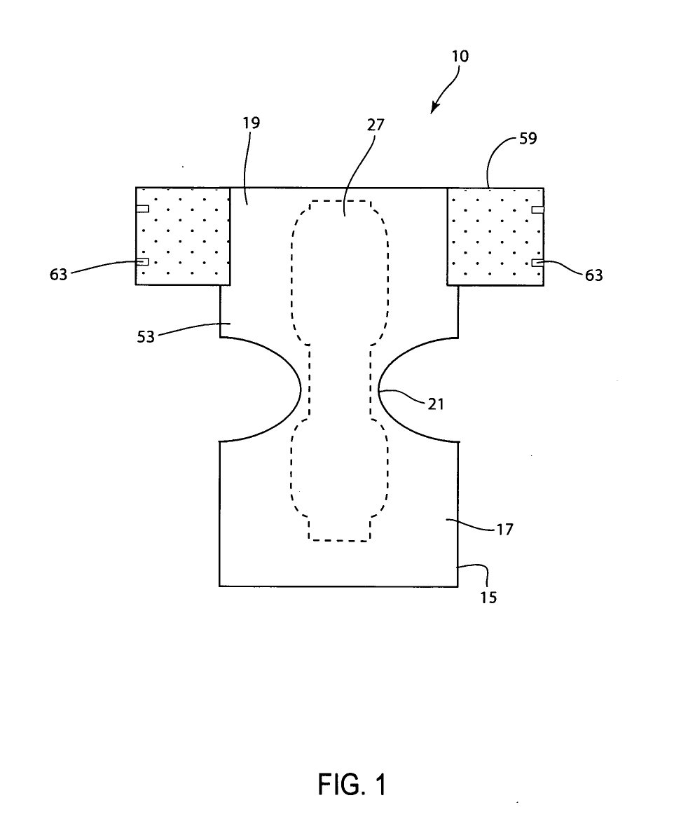

FIG. 1 illustrates an absorbent disposable bariatric diaper in the assembled state. An absorbent bariatric-style diaper of the kind which is intended for one-time use only and comprises of a spunbond polypropylene non-woven top sheet, an absorbent core containing fluff and super absorbent polymer, a fluid-impervious microporous polypropylene film backsheet that covers at minimal the absorbent core at it's widest point, a spunbond polypropylene non-woven backsheet layer that is glued with hotmelt to the fluid-impervious backsheet and the overlaying non-woven top sheet. The diaper will also contain standing leg-cuffs and elastomeric side panels (air-permeable) that are attached to the non-woven wings by a conventional fixation method (ultrasonic bonding, hotmelt adhesive, or heat seal bond). The problem with current Bariatric Briefs is that the design addresses waist size (girth) but does not address the issue of product length (rise). Product length (rise) is as important as waist size in that, as a person's body size increases in the waist dimension, the distance between the center line of the crotch and the normal beltline also increases, typically proportionately to waist increase. This increase in the crotch to beltline measurement can be symmetrical or asymmetrical, dependant on body build. As a result, caregivers using the current design product are often forced to rotate the product forward or backward to accommodate body type in an attempt to provide adequate coverage of the pubic area or posterior. This rotation repositions the core relative to the body and can compromise the products ability to provide proper incontinence protection. The improved design provides additional product length (rise) which allows the product to be positioned on the body of an obese person in the same manner as a non-obese person—this eliminates the rotation of the product on the body and allows for optimal positioning of the absorbent core to relative to the body. This increase in rise is accomplished by adding length to the front and back of the product. In doing so, the product now fits on a person in a near normal fashion relative to the person's beltline. In instances of severe obesity this results in the front of the product extending up and under the wearers pannis. This places plastic between skin folds which can cause an increase in skin temperature and increased skin moisture. These conditions can result in increased skin breakdown. To address this issue, this product has been designed with all breathable components in this region to optimize air flow and it has been coated on both sides of the product with an antimicrobial/antifungal to help mange skin health in the skin fold areas. The design of the diaper is such that the rise on the front and back of the diaper can be significantly longer than normal diapers, from about 3 inches to about 12 inches longer. The diaper design also incorporates from about 5 inches to about 12 inches long elastic panels containing mechanical hook-style tape tabs which forms a large waist opening of 100 inches+(2540 mm+) inch waist size. Other features of the diaper include microporous film (fluid-impervious back sheet), perforated/aperatured elastic side panels, and air-permeable non-woven panels which form a 100% breathable diaper. The last feature is the non-woven top sheet and the non-woven backsheet are coated with an antimicrobial/anti-fungal compound (AEGIS) that inhibits or aids in the reduction of bacterial/fungi (micro-organisms) to grow on the surfaces of the diaper. Thus both sides of the diaper are coated with the anti-fungal/antimicrobial material. One such antimicrobial/anti-fungal product that works particularly well for this product is sold under the trademark AEGIS. This inhibits skin breakdown and odor inside the diaper and also on the outside of the diaper where the diaper comes in contact with the wearer's skin folds, pannis, and hip folds. Typically, these are the areas where odor is produced and skin breakdown occurs due to the moist environment. With the anti-fungal/antimicrobial coating on the diaper, the diaper is essentially semi sterile when it is removed from its packaging.

This invention is directed to a bariatric style diaper or incontinent brief which is intended for a one-time use by the wearer. The features of the invention will be more readily understood by referring to the attached drawings in connection with the following description.

The diaper or brief 10 of the present invention has a non-woven top sheet 15 that is positioned to be adjacent the user of the diaper. The non-woven top sheet 15 has a front portion 17, a back portion 19 and a center portion 21. The top sheet has a longitudinal axis 23 that extends from the front portion to the rear portion of the diaper 10. The front portion 17 is disposed to be positioned adjacent the front of the user and the back portion 19 is disposed to be positioned adjacent the back of the user.

An absorbent core 27 containing fluff 29 and a super absorbent polymer 31 is positioned adjacent the top sheet 15. The absorbent core is disposed along the longitudinal axis of the top sheet. The absorbent core extends from the front portion 17 to the back portion 19 of the top sheet 15. A fluid impervious backsheet 35 is positioned on the side of the absorbent core 27 that is opposite to the side where the top sheet 15 is positioned. The backsheet can be a mircoporous material that can allow vapor to pass through backsheet while preventing the passage of liquid through the back sheet. The fluid impervious backsheet covers at a minimum the absorbent core at its widest point. The backsheet has a front section 39 and a back section 41 that are in an opposed relationship with the front and back portions of the top sheet 15.

A breathable non-woven layer 45 is secured to the fluid impervious backsheet 35. The non-woven layer 45 is also secured to the portions of the non-woven top sheet 15 that extends beyond the absorbent core 27. The non-woven layer 45 has a top portion 47 and a bottom portion 49. The top portion 47 is in opposed substantially aligned relationship with the front portion 17 of the top sheet 15 and the bottom portion 49 is in opposed substantially aligned relationship with the back portion 19 of the top sheet 15.

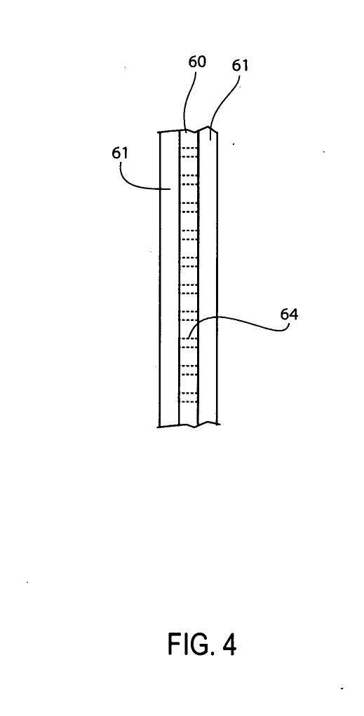

Wings 53 of spunbond non-woven polypropylene material extend from each side of the top and bottom portions of the non-woven layer 45. Air-permeable elastic side panels 59 extend from each of the wings 53 that extend from the edges of the top and bottom portions of the non-woven layer 45. The side panels 59 are attached to the non-woven wings by conventional methods such as ultrasonic bonding, hot melt adhesives or a heat seal bond. A very important feature to be expanded on is the elastic-breathable side panels. As show in FIG. 4 the elastic side panels are made with an elastomeric film 60 that is sandwiched between two non-woven layers 61. These layers are typically bonded together by thermal bonds (embossed-type patterns) which allows the composite to be stretchable and retractable. Our panels are produced in a way that are fully stretchable but are also perforated/apertured 64 to allow air-flow. Air permeability is high, where air permeability tested per ASTM D-737 is greater than 10 m³/m²/min. A typical design for the invention has an elastic composite for the elastic side panels containing from about 15 to about 45 holes/sq. inch with approximately 30 holes/square inch being preferred where holes are from about 0.5 mm to about 2.5 mm outside diameter in size with holes being about 1.75 mm being preferred in a semi-stretched mode (approx. 10-25% condition). Mechanical hook-style tape tabs 63 are secured to the elastomeric side panels 59 that extend from the bottom portion 46 of the non-woven layer 45. The tape tabs are designed to engage the wings 53 that extend from the top portion 47 of the non-woven layer 45 to secure the diaper around the user. The diaper will also contain standing leg cuffs 65 as is well known in the diaper art.

The non-woven top sheet 15, the fluid impervious backsheet 35, the non-woven layer 45, the wings 53 and the elastomeric side panels 59 are all made from materials that are breathable. These materials allow air and moisture vapor to pass through the materials in the areas that are outside of the absorbent core 27. The breathability factor for the materials used in the diaper 10 reduces the retention of moisture on the user's skin and also reduces the retention of heat under the diaper. Accordingly, a more comfortable breathable diaper is provided for the user. The elastomeric side panels 59 and the wings 53 can also be sized so that the diaper 10 can be used with subjects that are normally to large for currently available diaper products. The wings 53 and elastomeric side panels 59 provide a very cost affective manner for increasing the useable size of the diaper 10. The wings and elastic side panels function as an expansion joint to retain the diaper in the desired location on the user. The elastic panels bias the diaper towards around the user, but allow for stretch as the user moves or changes position. Thus keeping the diaper in position without being uncomfortable. If the traditional fixed securing tabs of a diaper are secured tightly enough to hold a bariatric diaper in place, sufficient force is applied to make the diaper uncomfortable. In addition, if the traditional tabs are so tight, there is a good chance the tabs or the diaper will fail when the user moves and places additional stress on the tabs.

The non-woven top sheet 15, the non-woven layer 45, the wings 53 and the elastomeric side panels 59 can be coated with an antimicrobial and anti-fugal compound that inhibits the growth of bacteria and fungi (micro-organisms) on the surfaces of the diaper. The antimicrobial and anti-fugal coating 71 inhibits skin break down and odor inside the diaper and also on the outside of the diaper where the diaper comes into contact with the skin of the user. An anti-microbial and anti-fungal compound sold under the trade name of AEGIS has been found to be very effective for this component of the diaper. In particular, skin folds, pannis and hip folds are places on large users where break down of the skin and odor can occur. The coating 71 on the inner and outer surfaces of the diaper 10, along with the breathable nature of this diaper construction functions to greatly reduce such problems that frequently occur with users of the diaper of the present invention.

The above detailed description of the present invention is given for explanatory purposes. It will be apparent to those skilled in the art that numerous changes and modifications can be made without departing from the scope of the invention. Accordingly, the whole of the foregoing description is to be construed in an illustrative and not a limitative sense, the scope of the invention being defined solely by the appended claims.

We claim:

1. A diaper for bariatric users comprising:
a topsheet that is positioned adjacent the user, the topsheet having a front portion that is positioned adjacent the front of the user and a back portion that is positioned adjacent the back of the user, a center portion of the topsheet is positioned between the front and rear portions, the topsheet having a longitudinal axis that extends from the front portion to the rear portion;
an absorbent core positioned adjacent the topsheet along the longitudinal axis of the top sheet; and
a fluid impervious backsheet positioned adjacent to the absorbent core and in spaced apart relationship with the top sheet;
the backsheet having a front section and a back section that are in opposed relationship with the front and back portions of the topsheet, the front portion and front section forming a front rise for the diaper, the back portion and back section forming a back rise for the diaper, at least one of the front or back rise being from about 3 inches to about 12 inches longer in a direction along the longitudinal axis than a traditional diaper whereby the diaper has a configuration that accommodates the shape of the bariatric user.

2. The diaper of claim 1 wherein the at least one rise is the front rise where the front of the diaper is expanded to accommodate the stomach of the user.

3. The diaper of claim 1 wherein the at least one rise is the back rise where the rear of the diaper is expanded to accommodate the rear of the user.

4. The diaper of claim 1 wherein the front rise and back rise are expanded to accommodate the bariatric user.

5. The diaper of claim 1 wherein a wing is formed on each side of the back sections of the backsheet.

6. The diaper of claim 5 wherein elastic panels are secured to the wings, the elastic panels having end portions that are disposed to be secured to the front section of the backsheet to maintain the diaper on the user.

7. The diaper of claim 6 wherein hook-style tabs are provided on each end of the elastic panels to secure the end portions to the front sections of the backsheet.

8. The diaper of claim 6 wherein the elastic panels are formed of an elastomeric film sandwiched between two non-woven

9. The diaper of claim 8 wherein the elastomeric film having a plurality of holes and is air permeable.

10. A diaper for large users comprising:
a breathable, non-woven topsheet that is positioned adjacent the user, the topsheet having a front portion that is positioned adjacent the front of the user and a back portion that is positioned adjacent the back of the user, a center portion of the topsheet is positioned between the front and rear portions, the topsheet having a longitudinal axis that extends from the front portion to the rear portion;
an absorbent core positioned adjacent the topsheet along the longitudinal axis of the top sheet;
a microporus fluid impervious breathable backsheet positioned adjacent to the absorbent care and in spaced apart relationship with the top sheet; the backsheet having a front section and a back section that are in opposed relationship with the front and back portions of the topsheet, a breathable non-woven layer that is secured to the fluid impervious backsheet, the non-woven layer having wings that extend from each side of the front and back sections of the fluid impervious backsheet; and
air permeable elastomeric side panels attached to the wings of non-woven material, the side panels and wings being disposed to allow the side panels on opposite sides of the backsheet to be secured together to secure the diaper to the user, the topsheet, backsheet, non-woven layer and elastomeric side panels being breathable.

11. The diaper of claim 1 wherein the topsheet and the non-woven layer on the backsheet, are coated with an antimicrobial compound.

12. The diaper of claim 10 wherein the topsheet and the non-woven layer on the backsheet are coated with an anti-fungal compound.

13. The diaper of claim 11 wherein the elastomeric side panels are coated with an antimicrobial compound.

14. The diaper of claim 12 wherein the elastomeric side panels are coated with an anti-fungal compound.

15. The diaper of claim 10 wherein the sides of the topsheet, the non-woven layer on the backsheet and the elastomeric side panels are coated with an antimicrobial and anti-fungal compound on the sides of these components that come into contact with the user.
